(19) United States
(12) Patent Application Publication
(10) Pub. No.: US 2024/0390155 A1
(43) Pub. Date: Nov. 28, 2024

(54) METATARSAL IMPLANT

(71) Applicant: Episurf IP-Management AB, Stockholm (SE)

(72) Inventors: Leif RYD, Malmö (SE); Johan JULIN, Älvsjö (SE)

(73) Assignee: Episurf IP-Management AB, Stockholm (SE)

(21) Appl. No.: 18/695,801

(22) PCT Filed: Sep. 27, 2022
(86) PCT No.: PCT/EP2022/076803

§ 371 Date: Mar. 26, 2024

(30) Foreign Application Priority Data
Sep. 27, 2021 (SE) 2151181-1

(51) Int. Cl. A61F 2/42; A61B 17/17; A61F 2/30

(52) U.S. Cl. CPC A61F 2/4225; A61B 17/1775; A61F 2/30942; A61F 2002/30617; A61F 2002/4233; A61F 2310/00407

(57) ABSTRACT

A metatarsal implant for repairing damage in a metatarsophalangeal joint of a patient is provided. The metatarsal implant is adapted to be attached to an implant receiving surface which has been created on a metatarsal head of the patient by sawing off sections of the metatarsal head, and an articulating surface of the metatarsal implant is designed to correspond to the curvature of a simulated healthy articulating surface of the undamaged metatarsal head at a site of diseased cartilage and/or bone. A bone contacting surface of the metatarsal implant is designed to correspond to the implant receiving surface, and the contour curvature of the articulating surface is generated based on a determined surface curvature of the cartilage and/or the bone in a predetermined area at the site of diseased cartilage and/or bone, to mimic the original, undamaged, articulating surface of the metatarsal head.

### TECHNICAL FIELD

The present disclosure relates generally to implants suitable for repairing damage in a toe of a patient, such as e.g. a big toe, especially damage in the metatarsophalangeal joint.

### BACKGROUND

The big toe (hallux) is vital for effective ambulation. The first metatarsophalangeal joint is a hallux joint that consists of the articulation between the first metatarsal head and the first proximal phalanges, as well as the articulation between the plantar aspect of the first metatarsal head and the sesamoid bones. The range of motion of the unaffected first metatarsophalangeal joint is largest in the sagittal plane and ranges between approximately 15° of plantar flexion to 75° dorsiflexion from standing position. Normal range of motion is approximately 65-100 degrees.

Hallux limitus (limited range of motion of the first metatarsophalangeal joint) is considered an earlier stage of progressive osteoarthritic disorder of the first metatarsophalangeal joint, due to acute or chronic injury to the first metatarsophalangeal joint, or of the rheumatoid arthritis, with genetic, autoimmune and inflammatory components. Hallux limitus may advance to the end-stage hallux rigidus, where the joint fuses. Hallux rigidus is associated with painful stiffness of the big toe, and is manifested by a decreased total arc of motion with near normal plantar flexion and a decreased dorsiflexion, secondary to a mechanical block by osteophytes (immature bone formation, also named bony spurs) and scarring of the plantar structures. The transversal motion of approximately 2 mm in the normal toe is 50% reduced in hallux rigidus, and this is thought to be due to the contracture of the collateral ligaments and the joint capsule.

The first metatarsophalangeal joint supports considerable mechanical loads during activity, even though it is not a direct weight-bearing articulation. High compressive loads are produced by associated muscle action. Stability of the first metatarsophalangeal joint is important for the stability of the medial column of the foot. Furthermore, the first metatarsophalangeal joint is subject to flexion, extension, abduction/adduction and pronation/supination forces. Shear stress is dissipated by dorsal gliding of the phalanx on the native metatarsal head, which spares the joint during gait, making it an ideal joint for hemiarthroplasty.

A number of different resurfacing implants for the metatarsophalangeal joint are known. Examples are shown e.g. in AU776010, WO2006052874, WO2009073924, US20100262254, US20120215320, and US20210059829. U.S. Pat. No. 9,888,931 describes a guide tool that is adapted for repair of damage in a finger or a toe.

WO2009073924 describes an embodiment of an endoprothesis for a metatarsophalangeal joint, and U.S. Pat. No. 5,774,203 describes a prosthetic joint for replacing the natural metatarsal-phalangeal-sesamoid joint of the toe. EP3013256 and US2019328548 describe guide tools that may be used for repairing damage in e.g. toes using a small implant placed in a hole that is drilled in the surface of the metatarsal bone.

### PROBLEMS WITH THE PRIOR ART

Implants for the first metatarsophalangeal joint often limit the dorsiflexion of the toe, and therefore often do not give the patient back a full range of motion. One reason for this is that they do not take full account of the sesamoid bones, which move around the metatarsal head as the toe is bent. Another reason may be that raw bone surfaces after osteophyte/bone spurs removal cause friction in the joint.

Therefore, there is a need for improved implants suitable for repairing damage in the big toe of a patient.

### SUMMARY

The above described problem is addressed by the claimed metatarsal implant for repairing damage in a metatarsophalangeal joint of a patient. The metatarsal implant is adapted to be attached to an implant receiving surface which has been created on a metatarsal head of the patient by sawing off sections of the metatarsal head so that the implant receiving surface becomes asymmetrical, in order to lock the metatarsal implant in a position where it cannot be rotated. A bone contacting surface of the metatarsal implant is designed to correspond to the implant receiving surface, and an articulating surface of the metatarsal implant is designed to correspond to the curvature of a simulated healthy articulating surface of the damaged metatarsal head at a site of diseased cartilage and/or bone. The contour curvature of the articulating surface is generated based on a determined surface curvature of the cartilage and/or the bone in a predetermined area at the site of diseased cartilage and/or bone, to mimic the original, undamaged, articulating surface of the metatarsal head. This enables the repairing of damage in a metatarsophalangeal joint of a patient with a metatarsal implant that takes full account of the sesamoid bones, and may also extend far enough to always interact with the sesamoid bones.

In embodiments, the articulating surface of the metatarsal implant comprises a positioning mark. This makes it easier to accomplish a correct rotational positioning of the metatarsal implant during surgery, which is important because the articulating surface of the metatarsal implant will in most situations not be rotationally symmetric. The positioning mark may e.g. be a rotational positioning mark, or an indication of a direction in relation to the anatomy of the joint.

In embodiments, the metatarsal implant comprises an implant peg extending from a bone contacting surface of the metatarsal implant. The implant peg may be designed for press-fit into a recess in the metatarsal bone. The use of press-fit (where the implant peg is slightly larger than the recess) secures the implant to the implant receiving surface on the metatarsal head. The implant peg may be tapered at the end, for easier insertion into the recess.

The above described problem is also addressed by the claimed metatarsophalangeal implant arrangement for repairing damage in a metatarsophalangeal joint of a patient. The metatarsophalangeal implant arrangement preferably comprises a phalangeal implant, comprising an articulating surface, and a metatarsal implant, adapted to be attached to an implant receiving surface which has been created on a metatarsal head by sawing off sections of the metatarsal head so that the implant receiving surface becomes asymmetrical, in order to lock the metatarsal implant in a position where it cannot be rotated. A bone contacting surface of the metatarsal implant is designed to correspond to the implant receiving surface, and the articulating surfaces of the phalangeal implant and the metatarsal implant are preferably designed to allow that they interact with each other when the implants are implanted into the metatarsophalangeal joint of the patient. The articulating surface of the metatarsal implant is preferably a metal, metal alloy or ceramic surface, and the articulating surface of the phalangeal implant is preferably not a metal, metal alloy or ceramic surface. This enables the repairing of damage in a metatarsophalangeal joint of a patient with an implant arrangement that avoids a metal-on-metal interface.

In embodiments, the articulating surface of the metatarsal implant comprises titanium or titanium alloy, titanium nitride, titanium niobium nitride, and/or a cobalt-chromium alloy. Such materials are very suitable for a metatarsal implant.

In embodiments, the articulating surface of the phalangeal implant comprises a polymer material, such as polyethylene, e.g. the polyethylene UHMWPE (e.g. cross-linked UHMWPE or vitamin E enhanced UHMWPE). This avoids a very hard surface, such as a metal, metal alloy or ceramic surface, interfacing with another very hard surface, creating e.g. a metal-on-metal interface between the implants. The main body of the phalangeal implant may be manufactured from metal, metal alloy or ceramic, but the articulating surface preferably comprises a polymer material, such as polyethylene, e.g. the polyethylene UHMWPE. If the bone contacting surface of the phalangeal implant is a non-porous metal, metal alloy or ceramic surface, it may be advantageous to coat the bone contacting surface with an osseointegrating and/or bioactive material, such as e.g. hydroxyapatite.

The metatarsal implant may be the above described metatarsal implant, but it may also be a standardized metatarsal implant, selected from a predefined set of standardized metatarsal implants having varying dimensions.

The above described problem is further addressed by the claimed metatarsal surgical kit (kit of surgical instruments). The metatarsal surgical kit preferably comprises: the above described metatarsal implant; at least one metatarsal saw guide comprising a contact surface configured to have a shape and contour that is designed to correspond to and to fit the actual contour of the metatarsal bone in a predetermined area of the metatarsal bone; and a metatarsal drill guide for drilling a recess for an implant peg extending from a bone contacting surface of the metatarsal implant, the metatarsal drill guide comprising a contact surface configured to have a shape and contour that is designed to correspond to and to fit an implant receiving surface on a metatarsal head.

In embodiments, the metatarsal surgical kit further comprises an insert tool configured to be used for attaching the metatarsal implant to the implant receiving surface on the metatarsal head, wherein the insert tool has an implant engaging portion that has a surface curvature that substantially corresponds to the surface curvature of the articulating surface of the metatarsal implant.

The above described problem is further addressed by the claimed metatarsophalangeal surgical kit (kit of surgical instruments) comprising: the above described metatarsophalangeal implant arrangement; at least one metatarsal saw guide comprising a contact surface configured to have a shape and contour that is designed to correspond to and to fit the actual contour of the metatarsal bone in a predetermined area of the metatarsal bone; a phalangeal guide tool comprising a contact surface configured to have a shape and contour that is designed to correspond to and to fit the actual contour of the bone in a predetermined area of the proximal phalanges; and one or more insert tools, configured to be used for attaching the metatarsal implant to an implant receiving surface on a metatarsal head, and/or attaching the phalangeal implant to an implant receiving surface on a proximal phalanges. Preferably, the saw guide for the metatarsal implant and/or the guide tool for the phalangeal implant comprise visual markings, so that they are visually distinct from each other. In this way, it will be clear to the surgeon which guide tool to use for which implant.

The above described problem is further addressed by the claimed metatarsal saw guide comprising a contact surface configured to have a shape and contour that is designed to correspond to and to fit the actual contour of the metatarsal bone in a predetermined area of the metatarsal bone, wherein the metatarsal saw guide comprises a plurality of different saw blade guides.

In embodiments, the metatarsal saw guide comprises one or more contact surface extensions that extend around at least a part of the metatarsal head.

The above described problem is also addressed by the claimed system for customizing a metatarsal implant for repairing damage in a metatarsophalangeal joint of a patient, where the metatarsal implant is adapted to be attached to an implant receiving surface which has been created on a metatarsal head of the patient by sawing off sections of the metatarsal head so that the implant receiving surface becomes asymmetrical, in order to lock the metatarsal implant in a position where it cannot be rotated. The system preferably comprises at least one processor configured to: obtain a three-dimensional image representation of the metatarsophalangeal joint of the patient based on medical images generated using a medical imaging system; determine damage to the metatarsophalangeal joint of the patient by analyzing medical images generated using a medical imaging system; and determine the shape and dimensions of a customized metatarsal implant suitable for repairing said determined damage, using said three-dimensional image representation of the metatarsophalangeal joint by designing a bone contacting surface of the metatarsal implant to correspond to the implant receiving surface, and generating the contour curvature of the articulating surface based on the determined surface curvature of the cartilage and/or the bone in a predetermined area at the site of diseased cartilage and/or bone, to mimic the original, undamaged, articulating surface of the metatarsal head. This enables the customizing of a metatarsal implant that takes full account of the sesamoid bones, and may also extend far enough to always interact with the sesamoid bones.

In embodiments, the at least one processor is configured to determine the shape and dimensions of the customized metatarsal implant by simulating a healthy articulating metatarsal surface at the site of the determined damage, including designing the surface of the customized metatarsal implant to match said simulated healthy articulating metatarsal surface.

The above described problem is further addressed by the claimed method for customizing a metatarsal implant for repairing damage in a metatarsophalangeal joint of a patient, where the metatarsal implant is adapted to be attached to an implant receiving surface on a metatarsal head of the patient. The method preferably comprises: obtaining a three-dimensional image representation of the metatarsophalangeal joint based on medical images generated using a medical imaging system; determining damage to the metatarsophalangeal joint by analyzing medical images generated using a medical imaging system; and determining the shape and dimensions of a customized metatarsal implant suitable for repairing said determined damage, using said three-dimensional image representation of the metatarsophalangeal joint. This enables the customizing of a metatarsal implant that takes full account of the sesamoid bones, and may also extend far enough to always interact with the sesamoid bones.

In embodiments, the determining of the shape and dimensions of the customized metatarsal implant involves simulating a healthy articulating metatarsal surface at the site of the determined damage, including designing the surface of the customized metatarsal implant to match said simulated healthy articulating metatarsal surface.

The above described problem is also addressed by a non-transitory machine-readable medium on which is stored machine-readable code which, when executed by a processor, controls the processor to perform any one of the above described methods.

The above described problem is further addressed by the claimed method for attaching a metatarsal implant to an implant receiving surface on a metatarsal head, for repairing damage in a metatarsophalangeal joint of a patient. The method preferably comprises: attaching at least one metatarsal saw guide to the metatarsal bone, the metatarsal saw guide comprising a contact surface configured to have a shape and contour that is designed to correspond to and to fit the actual contour of the metatarsal bone in a predetermined area of the metatarsal bone; creating an implant receiving surface on the metatarsal head by sawing the implant receiving surface, using the at least one metatarsal saw guide; removing the at least one metatarsal saw guide from the metatarsal bone; using a metatarsal implant dummy for verifying that the implant receiving surface has the correct size and shape for receiving the metatarsal implant; attaching a metatarsal drill guide to the metatarsal bone, the metatarsal drill guide comprising a contact surface configured to have a shape and contour that is designed to correspond to and to fit the implant receiving surface on the metatarsal head; drilling a recess for an implant peg extending from a bone contacting surface of the metatarsal implant; removing the metatarsal drill guide from the metatarsal bone; placing the metatarsal implant on the implant receiving surface; pressing the metatarsal implant to the implant receiving surface using an insert tool; and removing the insert tool.

In embodiments, the method comprises applying an adhesive, such as e.g. bone cement, on the implant receiving surface, and/or on a bone contacting surface of the metatarsal implant, before placing the metatarsal implant on the implant receiving surface. The bone contacting surface may have a structure that improves osseointegration, such as e.g. a lattice structure or a random lattice structure.

In embodiments, the method comprises making a marking on the cartilage at the side of the implant receiving surface on the metatarsal head, in order to ensure a correct rotational positioning of the implant.

The contact surface of the metatarsal saw guide mainly contacts the metatarsal bone, so it may also be called a bone contact surface.

The above referenced metatarsophalangeal joint is preferably the first metatarsophalangeal joint, but other metatarsophalangeal joints of a patient are also conceivable.

The medical imaging system may e.g. be a magnetic resonance imaging (MRI) system, an x-ray imaging system, an ultrasonic imaging system, a fluoroscopic imaging system and/or a computer tomography (CT) system, e.g. CBCT. The medical images may be a number of images in a series captured during a process of scanning through different layers of the anatomical joint or part of it using a medical imaging system.

The processor may in some embodiments comprise several different processors which together perform the claimed functions.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for customizing a metatarsal implant, in accordance with one or more embodiments described herein.

FIGS. 2a-b illustrate a metatarsophalangeal implant arrangement for repairing damage in a metatarsophalangeal joint, in accordance with one or more embodiments described herein.

FIGS. 3a-d illustrate embodiments of a metatarsal implant, in accordance with one or more embodiments described herein.

FIGS. 4a-b illustrate the creation of an implant receiving surface suitable for receiving a metatarsal implant on a first metatarsal head, in accordance with one or more embodiments described herein.

FIG. 4c illustrates a first metatarsal bone comprising an implant receiving surface suitable for receiving a metatarsal implant on a first metatarsal head, in accordance with one or more embodiments described herein.

FIGS. 4d-e illustrate embodiments of a metatarsal implant positioned on a first metatarsal head, in accordance with one or more embodiments described herein.

FIGS. 5a-h illustrate embodiments of a metatarsal saw guide, in accordance with one or more embodiments described herein.

FIG. 6a illustrates a metatarsal implant dummy, in accordance with one or more embodiments described herein.

FIG. 6b illustrates a metatarsal drill guide, in accordance with one or more embodiments described herein.

FIG. 7 is a schematic flow diagram for a method for customizing a metatarsal implant, in accordance with one or more embodiments described herein.

FIG. 8 is a schematic flow diagram for a method for attaching a metatarsal implant to a metatarsal head, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

Implants for the first metatarsophalangeal joint often limit the dorsiflexion of the toe, and therefore do not give the patient back a full range of motion. One reason for this is that they do not take full account of the sesamoid bones, which move around the metatarsal head as the toe is bent. Unless the sesamoid gliding path is perfectly smooth, there is always a risk that the sesamoid bones will lock against the implant. Another reason may be that raw bone surfaces after osteophyte/bone spurs removal cause friction in the joint.

The present disclosure relates generally to implants suitable for repairing damage in a toe of a patient, such as e.g. a big toe, especially damage in the first metatarsophalangeal joint. Embodiments of the disclosed solution are presented in more detail in connection with the figures.

### System Architecture

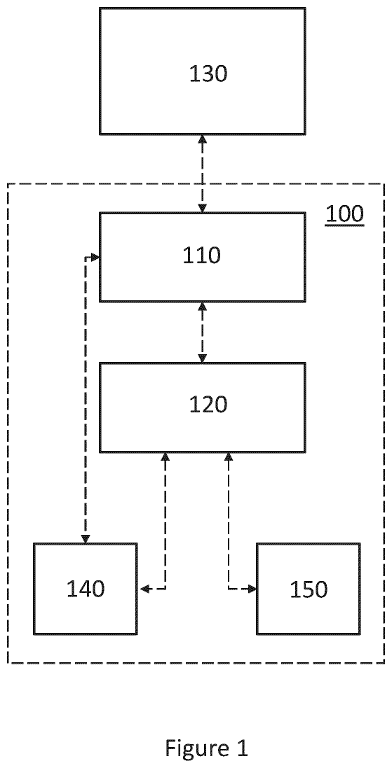

FIG. 1 shows a schematic view of a system 100 for customizing a metatarsal implant for repairing damage in a metatarsophalangeal joint of a patient. The metatarsal implant is adapted to be attached to an implant receiving surface which has been created on a metatarsal head of a patient by sawing off sections of the metatarsal head. According to embodiments, the system 100 comprises a display 140, at least one manipulation tool 150, and a storage media 110, configured to receive and store image data and parameters. In some embodiments, the system 100 is communicatively coupled to a medical imaging system 130. The medical imaging system 130 may be configured to capture or generate medical images, e.g. radiology images such as X-ray images, ultrasound images, computed tomography (CT), e.g. CBCT, images, nuclear medicine including positron emission tomography (PET) images, and magnetic resonance imaging (MRI) images. The storage media 110 may be configured to receive and store medical images from the medical imaging system 130. In embodiments, medical images are uploaded into the storage media 110 by personnel at a medical care facility, preferably the medical care facility where the medical imaging takes place. Medical images may however also be uploaded into the storage media 110 by another medical care facility, or by other authorized personnel. The uploading of the medical images may also be an automatic uploading directly from one system to another.

In one or more embodiments, the system 100 comprises at least one processor 120 configured to: obtain a three-dimensional image representation of a metatarsophalangeal joint of a patient based on medical images generated using a medical imaging system 130; determine damage to the metatarsophalangeal joint of the patient by analyzing medical images generated using a medical imaging system 130; and determine the shape and dimensions of a customized metatarsal implant 300 suitable for repairing said determined damage, using said three-dimensional image representation of the metatarsophalangeal joint, wherein the contour curvature of the articulating surface 310 is generated based on the determined surface curvature of the cartilage and/or the bone in a predetermined area at the site of diseased cartilage and/or bone, to mimic the original, undamaged, articulating surface of the metatarsal head 410.

In one or more embodiments, the at least one processor 120 is configured to determine the shape and dimensions of the customized metatarsal implant 300 by simulating a healthy articulating metatarsal surface at the site of the determined damage, including designing the surface of the customized metatarsal implant 300 to match the simulated healthy articulating metatarsal surface. The determination of the shape and dimensions of a customized metatarsal implant 300 preferably involves designing an implant surface that corresponds to a 3D image of a simulated healthy cartilage surface.

In embodiments, the at least one processor 120 is configured to also output the shape and dimensions of the customized metatarsal implant 300 as parameters for manufacturing said customized metatarsal implant 300.

The at least one processor 120 may for example be a general data processor, or other circuit or integrated circuit capable of executing instructions to perform various processing operations. The at least one processor 120 may in some embodiments comprise several different processors 120 which together perform the claimed functions. In the same way, the storage media 110 may in some embodiments comprise several different storage media 110 which together perform the claimed functions.

The display 140 may be configured to receive image data for display via the processor 120, and/or to retrieve image data for display directly from the storage media 110, possibly in response to a control signal received from the processor 120 or the at least one manipulation tool 150.

The processor 120 may further be configured to perform any or all of the method steps of any or all of the embodiments presented herein.

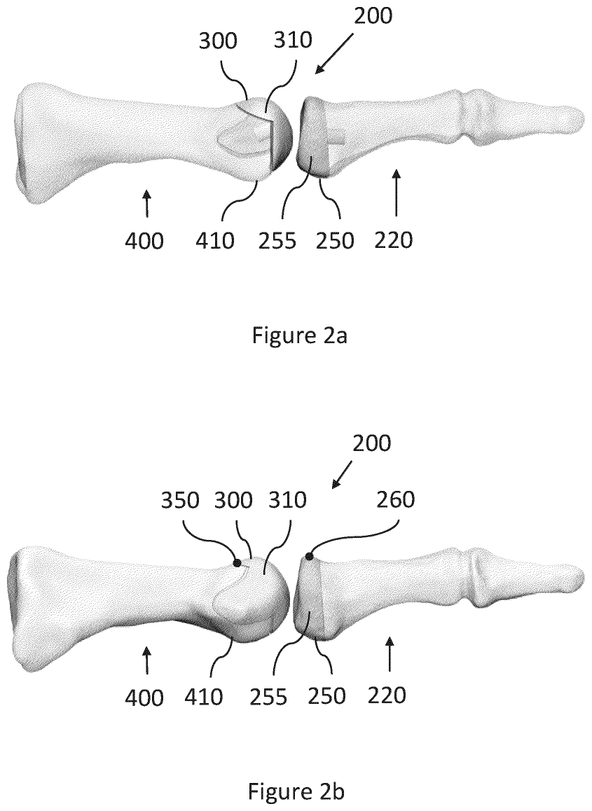

FIGS. 2a-b illustrate a metatarsophalangeal implant arrangement 200 for repairing damage in a first metatarsophalangeal joint of a patient, comprising a metatarsal implant 300 and a phalangeal implant 250. The implants 250, 300 are illustrated as implanted into the first metatarsophalangeal joint, with the metatarsal implant 300 attached to the first metatarsal head 410 and the phalangeal implant 250 attached to the proximal end of the first proximal phalanges 220. The implants 250, 300 comprise articulating surfaces 255, 310 that are designed to allow them to interact with each other when the implants 250, 300 are implanted into the first metatarsophalangeal joint of the patient.

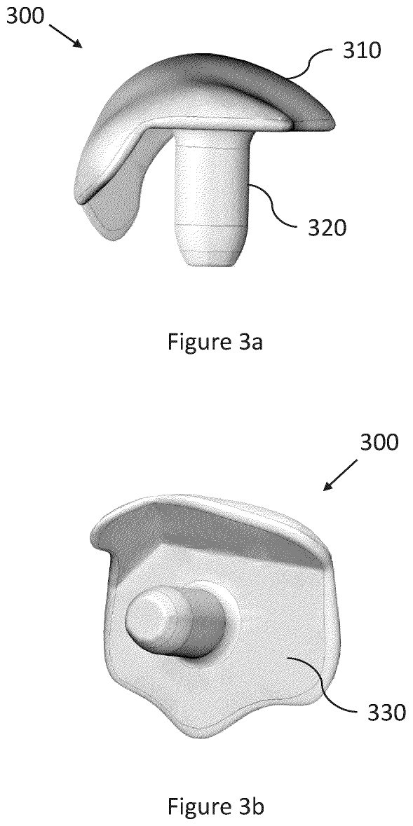

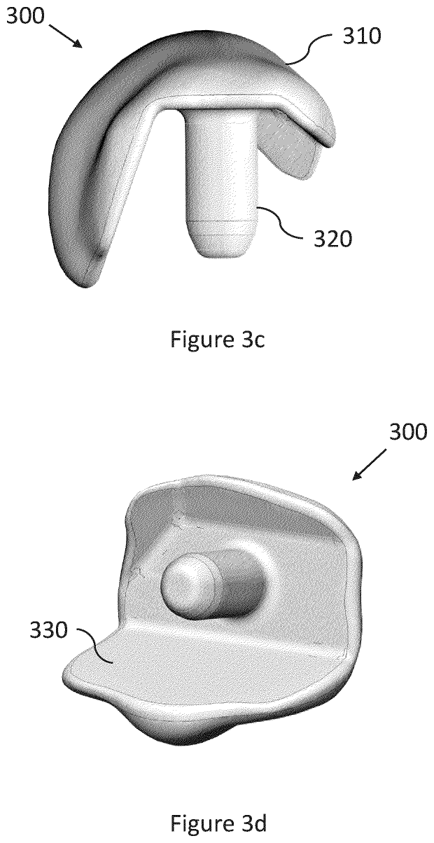

FIGS. 3a-d illustrate a metatarsal implant 300 for repairing damage in a first metatarsophalangeal joint of a patient. The metatarsal implant 300 is preferably configured to be attached to an implant receiving surface 420 which has been created on a metatarsal head 410 by sawing off sections of the metatarsal head 410. Since there is often damage on more than one side of the metatarsal head 410, it is often desirable for the metatarsal implant 300 to be shaped so that it encloses the end of the metatarsal head 410. There may e.g. be osteophytes that need to be removed from the metatarsal head 410, and in that case the metatarsal implant 300 preferably encloses the portions of the metatarsal head 410 where such osteophytes have been removed.

The metatarsal implant 300 may be manufactured in a number of different ways, including 3D printing. The articulating surface 310 of the metatarsal implant 300 is preferably a metal, metal alloy or ceramic surface, e.g. comprising titanium (Ti), titanium alloy, titanium nitride (TiN), titanium niobium nitride (TiNbN), and/or a cobalt-chromium (CoCr) alloy. It is preferably polished to a perfectly smooth surface, with a very low surface roughness, to lower the risk of a sesamoid bone locking against the articulating surface 310 of the metatarsal implant 300. The bone contacting surface 330 of the metatarsal implant 300 may be coated with an osseointegrating and/or bioactive material, such as e.g. hydroxyapatite. The bone contacting surface 330 of the metatarsal implant 300 may alternatively be coated with titanium (Ti), titanium alloy, titanium nitride (TiN), or titanium niobium nitride (TiNbN), This reduces the need for using an adhesive for securing the metatarsal implant 300 to the metatarsal head 410, but an adhesive (such as e.g. bone cement) may be used anyhow. The bone contacting surface 330 may have a structure that improves osseointegration, such as e.g. a lattice structure or a random lattice structure.

In order to avoid a very hard surface, such as a metal, metal alloy or ceramic surface, interfacing with another very hard surface, creating e.g. a metal-on-metal interface, the phalangeal implant 250 preferably has an articulating surface 255 that is not a metal, metal alloy or ceramic surface. The articulating surface 255 of the phalangeal implant 250 is preferably a polymer surface, e.g. a surface of polyethylene, e.g. the polyethylene UHMWPE (e.g. cross-linked UHMWPE or vitamin E enhanced UHMWPE). Preferably, the whole phalangeal implant 250 is manufactured from the same polymer material, since this simplifies the manufacturing process.

The main body of the phalangeal implant 250 may be manufactured from metal, metal alloy or ceramic, but the articulating surface 255 preferably comprises a polymer material, such as polyethylene, e.g. the polyethylene UHMWPE. If the bone contacting surface of the phalangeal implant 250 is a non-porous metal, metal alloy or ceramic surface, comprising e.g. titanium (Ti) or titanium alloy, titanium nitride (TiN), titanium niobium nitride (TiNbN), and/or a cobalt-chromium (CoCr) alloy, it may be advantageous to coat the bone contacting surface with an osseointegrating and/or bioactive material, such as e.g. hydroxyapatite. The bone contacting surface 330 of the metatarsal implant 300 may alternatively be coated with titanium (Ti), titanium alloy, titanium nitride (TIN), or titanium niobium nitride (TiNbN), This reduces the need for using an adhesive for securing the phalangeal implant 250 to the proximal phalanges 220, but an adhesive (such as e.g. bone cement) may be used anyhow.

The metatarsal implant preferably has an implant peg 320 extending from a bone contacting surface 330, and the phalangeal implant 250 preferably also has an implant peg extending from a bone contacting surface. The pegs of the implants 250, 300 are preferably designed for press-fit into recesses in the bone. The pegs of one or both of the implants 250, 300 may be tapered at the end, for easier insertion into the recess. If an adhesive such as e.g. bone cement is used, it may not be necessary for the peg of the implant 250, 300 to be designed for press-fit into the recess. The use of press-fit (where the implant peg is slightly larger than the recess) secures the implant 250, 300 to the implant receiving surface 420 on the metatarsal head 410 regardless of whether an adhesive such as bone cement is used, but the combination of press-fit and adhesive of course secures the implant 250, 300 even more to the implant receiving surface 420. One or both of the implants 250, 300 may comprise one or more recesses for bone cement in the peg, which secures the implant 250, 300 even further.

The phalangeal implant 250 and/or the metatarsal implant 300 may also comprise a positioning mark 260, 350, preferably positioned on the articulating surface 255, 310. This makes it easier to accomplish a correct rotational positioning of the implant during surgery, which may be important because the articulating surface of the implant will in most situations not be rotationally symmetric. The positioning mark 260 may e.g. be a rotational positioning mark, or an indication of a direction in relation to the anatomy of the joint.

The surface curvature of the articulating surface 310 of the metatarsal implant 300 preferably corresponds as closely as possible to the surface curvature of the undamaged metatarsal head 410, and in the embodiment of FIGS. 3c-d also extends far enough to always interact with the sesamoid bones, even when the toe is straight. In this way, full account is taken of the sesamoid bones, which move around the metatarsal head as the toe is bent. This increases the possibility of the patient being given back a full range of motion.

By analyzing the surface curvature of the cartilage and/or the bone in a predetermined area comprising and surrounding the site of diseased cartilage, it is possible to simulate a healthy articulating surface of the damaged metatarsal head 410 and mimic the original, undamaged, articulating surface of the metatarsal head 410. The image data may be analyzed in a data processing system to identify and determine physical parameters for the cartilage and/or bone damage. The physical parameters to be determined may comprise the presence, the location and the size and shape of the cartilage and/or bone damage, as well as curvature of the surface contour of the cartilage or the bone in an area of the cartilage and/or bone damage.

When such a healthy articulating metatarsal surface has been simulated, it is possible to design an individualized metatarsal implant 300 with an articulating surface 310 that corresponds to the simulated healthy metatarsal surface.

However, it is also possible to select the best matching predefined surface from a limited number of different predefined surfaces. This enables the use of standardized metatarsal implants 300. In this way, a set of standardized metatarsal implants 300 of different dimensions may be manufactured and stored, to be later used for repairing damage in the metatarsophalangeal joint.

A standardized metatarsal implant 300 may in this case be selected from a predefined set of standardized metatarsal implants 300 having varying dimensions. The predefined set of standardized metatarsal implants 300 is preferably created by analyzing dimensional data from stored images of the metatarsal head 410 from a large number of different patients. The standardized metatarsal implant 300 should be selected as a standardized metatarsal implant 300 having dimensions that match the shape of the metatarsal head 410 of the patient, thereby making it suitable for repairing the determined damage. A 3D model of the metatarsophalangeal joint, visualizing the determined damage, may be used in order to determine which standardized metatarsal implant 300 is the best fit for the metatarsal head 410 of the patient.

However, even if it is possible to use a standardized metatarsal implant 300, there will always be cases where it cannot be ascertained that a standardized metatarsal implant 300 will really fit the implant receiving surface 420 on the metatarsal head 410, and repair the damage while taking full account of the sesamoid bones. In order to ascertain that the metatarsal implant 300 will really fit the implant receiving surface 420 on the metatarsal head 410, and repair the damage while taking full account of the sesamoid bones, it is necessary to design an individualized metatarsal implant 300 with an articulating surface 310 that corresponds to the simulated healthy metatarsal surface, which may also extend far enough to always interact with the sesamoid bones.

In the same way as for the metatarsal implant 310, the surface curvature of the articulating surface 255 of the phalangeal implant 250 preferably corresponds as closely as possible to the surface curvature of the undamaged proximal phalanges 220.

By analyzing the surface curvature of the cartilage and/or the bone in a predetermined area comprising and surrounding the site of diseased cartilage, it is possible to simulate a healthy articulating surface of the damaged proximal phalanges 220 and mimic the original, undamaged, articulating surface of the proximal phalanges 220. The image data may be analyzed in a data processing system to identify and determine physical parameters for the cartilage and/or bone damage. The physical parameters to be determined may comprise the presence, the location and the size and shape of the cartilage and/or bone damage, as well as curvature of the surface contour of the cartilage or the bone in an area of the cartilage and/or bone damage.

When such a healthy articulating metatarsal surface has been simulated, it is possible to design an individualized phalangeal implant 250 with an articulating surface 255 that corresponds to the simulated healthy phalangeal surface.

However, it is also possible to select the best matching predefined surface from a limited number of different predefined surfaces. This enables the use of standardized phalangeal implants 250. In this way, a set of standardized phalangeal implants 250 of different dimensions may be manufactured and stored, to be later used for repairing damage in the metatarsophalangeal joint.

A standardized phalangeal implant 250 may in this case be selected from a predefined set of standardized phalangeal implants 250 having varying dimensions. The standardized phalangeal implant 250 should be selected as a standardized phalangeal implant 250 having dimensions that match the shape of the proximal end of the proximal phalanges 220 of the patient, thereby making it suitable for repairing the determined damage. A 3D model of the metatarsophalangeal joint, visualizing the determined damage, may be used in order to determine which standardized phalangeal implant 250 is the best fit for the proximal end of the proximal phalanges 220 of the patient.

However, even if it is possible to use a standardized phalangeal implant 250, there will always be cases where it cannot be ascertained that a standardized phalangeal implant 250 will really fit the implant receiving surface on the proximal phalanges, and be designed to interact perfectly with the metatarsal implant 300. In order to ascertain that the phalangeal implant 250 will really fit the implant receiving surface on the proximal phalanges, and be designed to interact perfectly with the metatarsal implant 300, it is necessary to design an individualized phalangeal implant 250 with an articulating surface 255 that corresponds to the simulated healthy phalangeal surface.

The metatarsal implant 300 may be used alone, or together with a phalangeal implant 250 in a metatarsophalangeal implant arrangement 200, as illustrated in FIGS. 2a-b. In such a metatarsophalangeal implant arrangement 200, it is preferred if either both the metatarsal implant 300 and the phalangeal implant 250 are individualized, or both the metatarsal implant 300 and the phalangeal implant 250 are standardized. However, it is also possible to combine an individualized metatarsal implant 300 with a standardized phalangeal implant 250, or a standardized metatarsal implant 300 with an individualized phalangeal implant 250.

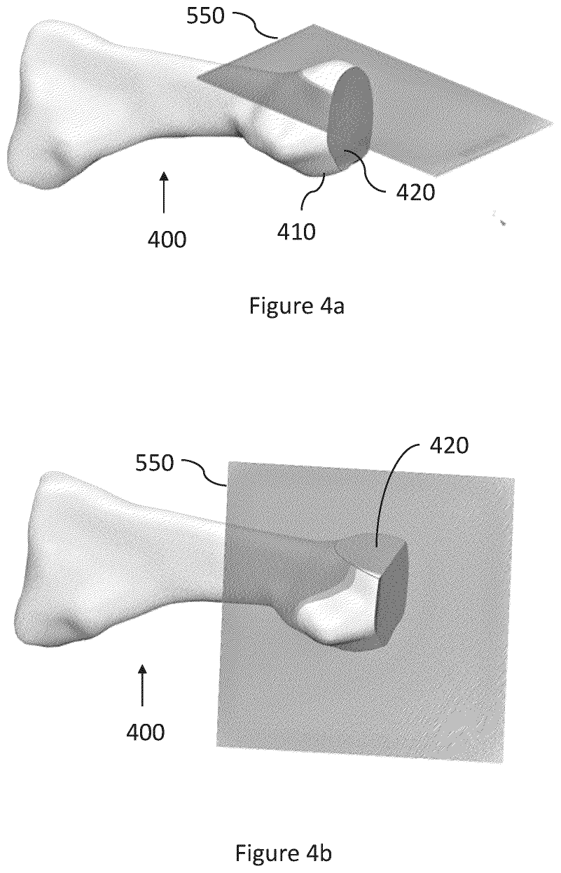

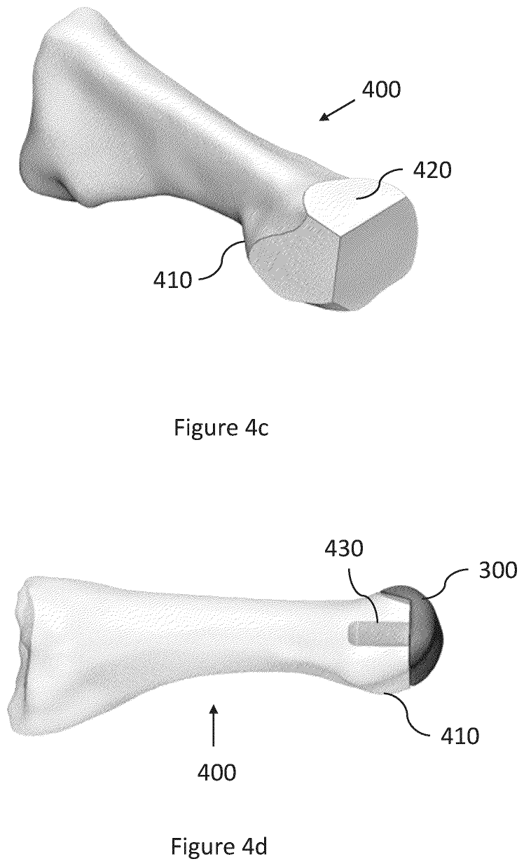

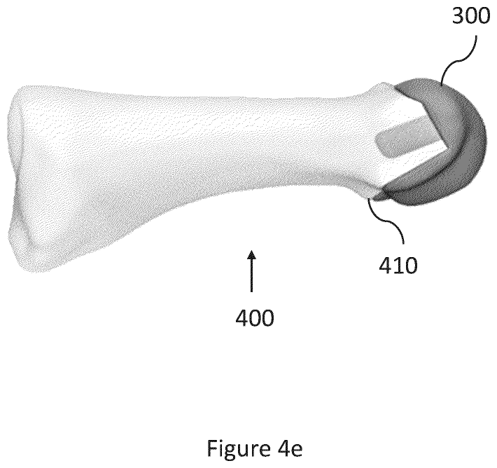

FIGS. 4a-b illustrate the creation of an implant receiving surface 420 suitable for receiving a metatarsal implant 300 on a metatarsal head 410, FIG. 4c illustrates a first metatarsal bone 400 comprising an implant receiving surface 420 suitable for receiving a metatarsal implant 300 on a first metatarsal head 410, and FIG. 4d-e illustrate embodiments of a metatarsal implant 300 positioned on a first metatarsal head 410.

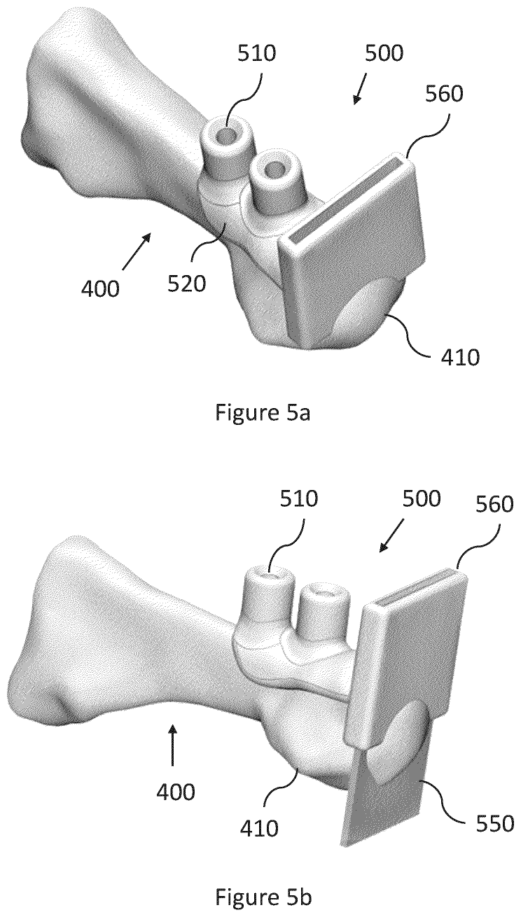

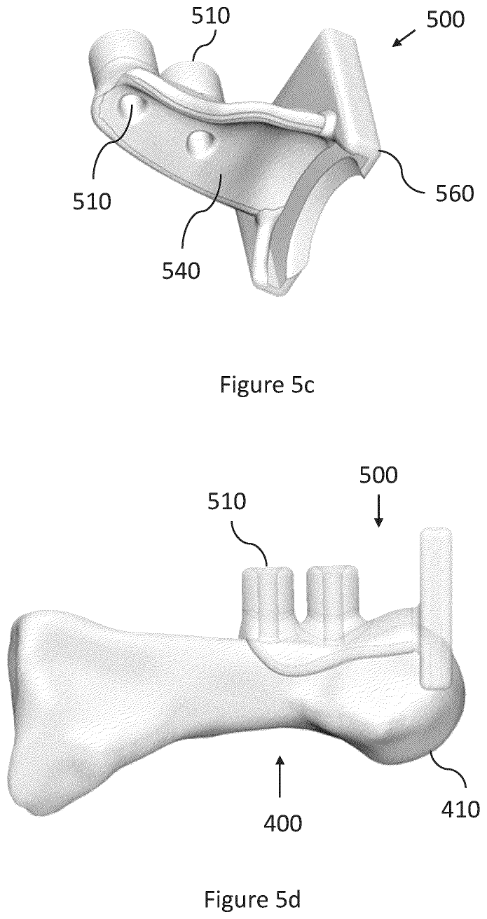

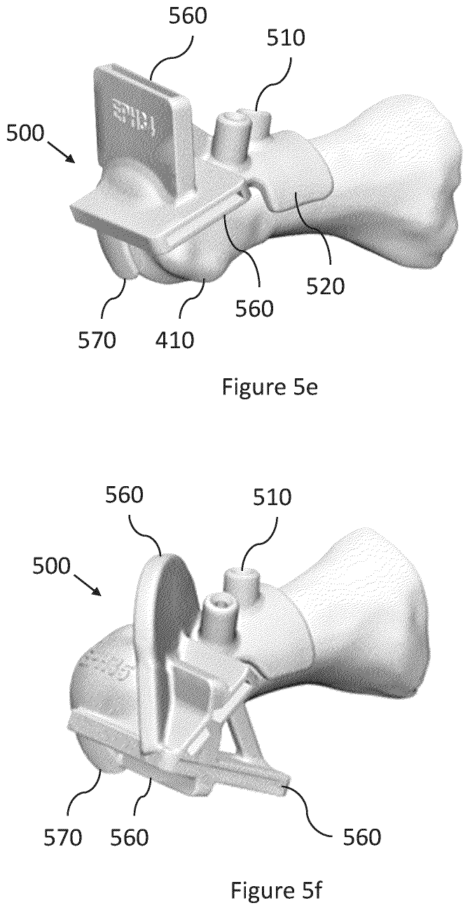

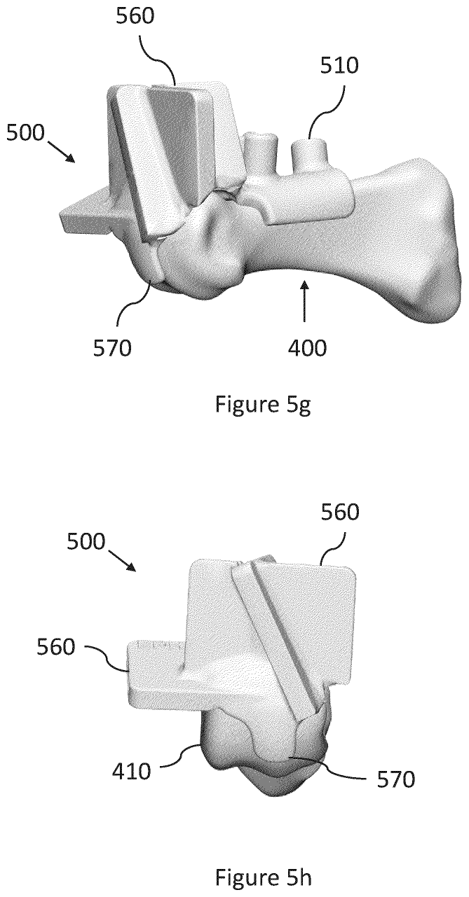

FIGS. 5a-h illustrate embodiments of a metatarsal saw guide for creating an implant receiving surface 420 on a metatarsal head 410. The saw guide 500 preferably has a contact surface 540 that has a shape and contour that is designed to correspond to and to fit the actual contour of the metatarsal bone 400 in a predetermined area of the metatarsal bone 400. The predetermined area is preferably located to allow the creation of an implant receiving surface 420 at the site of diseased cartilage. Thereby, the contact surface 540 of the saw guide 500 corresponds to and fits to the actual contour of the surface of the metatarsal bone 400 at the selected location. The whole contact surface 540 does not have to correspond to the actual contour curvature of the metatarsal surface at the selected location, it is enough if the contact surface 540 comprises at least three contact points, so that the metatarsal saw guide 500 will have a stable mounting in the correct position at the selected location. This helps ensuring that the implant receiving surface 420 will be created in the exact position of the determined damage. Such contact points are preferably chosen to provide maximum support and positional stability for the metatarsal saw guide 500.

The contact surface 540 of the metatarsal saw guide 500 may be stabilized by comprising one or more contact surface extensions 570 that extend around at least a part of the metatarsal head 410, as illustrated in FIGS. 5e-h. Such contact surface extensions 570 help ensuring that the metatarsal saw guide 500 is positioned in the exact desired position on the metatarsal bone 400, and also increases the stability of the metatarsal saw guide 500 on the metatarsal bone 400. Since the metatarsal bone 400 is quite a small bone, exact positioning and stability of the metatarsal saw guide 500 is more important than for larger bones.

The contact surface 540 of the metatarsal saw guide 500 may be further stabilized by being attached to the metatarsal surface 400 with one or more nails, rivets, wires or similar attachment means that are inserted into through-holes 510 in the metatarsal saw guide 500. Such additional attachment gives additional support and stability, and enables the contact surface 540 of the metatarsal saw guide 500 to be as small as possible, which is especially important for the metatarsal bone 400, since it is quite small.

The contact surface of the metatarsal saw guide 500 mainly contacts the metatarsal bone, so it may also be called a bone contact surface.

The metatarsal saw guide 500 illustrated in FIGS. 5a-d is arranged to receive a saw blade 550 for sawing off the end of the metatarsal head 410. The metatarsal saw guide 500 illustrated in FIGS. 5e-h is arranged to receive saw blades 550 in different positions for sawing off different sections of the metatarsal head 410, in order to create an asymmetrical implant receiving surface 420, which locks the metatarsal implant 300 in a position where it cannot be rotated. The metatarsal saw guide 500 is preferably arranged so that the same base section 520 can receive different saw blade guides 560, e.g. by the saw blade guides 560 being clicked onto the base section 520, without the base section 520 having to be removed from the metatarsal bone 400. In the embodiment illustrated in FIGS. 5e-h, the metatarsal saw guide 500 is instead a “multiguide”, that comprises a number of different saw blade guides 560. The same metatarsal saw guide 500 could thus be used for receiving saw blade guides 560 for the saw blades 550 for sawing on all desired sides of the metatarsal head 410, in order to create an asymmetrical implant receiving surface 420, which locks the metatarsal implant 300 in a position where it cannot be rotated. Alternatively, a number of different metatarsal saw guides 500 may be used. It is then preferred if the same nails, rivets, wires or similar attachment means are used for all the different metatarsal saw guides 500, so that unnecessary drilling of holes in the metatarsal bone 400 can be avoided.

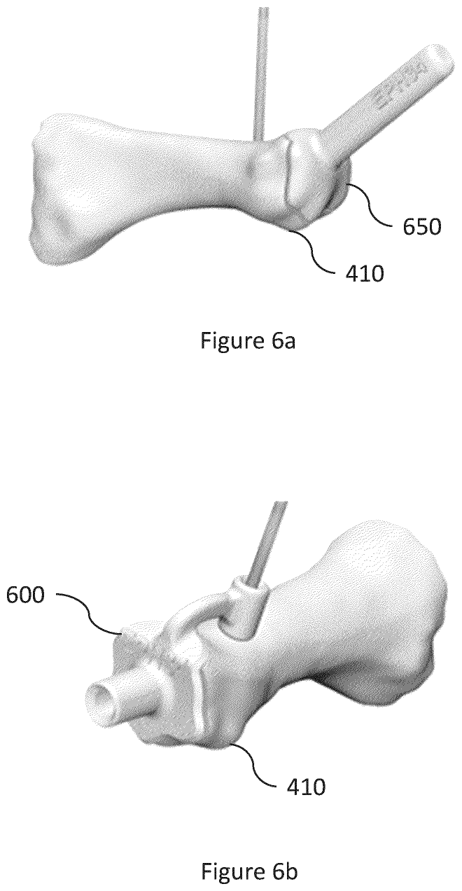

When the implant receiving surface 420 has been created on the metatarsal head 410 using the saw guide 500, a metatarsal implant dummy 650 (as illustrated in FIG. 6a) is preferably used for verifying that the implant receiving surface 420 has the correct size and shape for receiving the metatarsal implant 300, before the recess for the implant peg 320 is drilled using a metatarsal drill guide 600 (illustrated in FIG. 6b). The metatarsal drill guide 600 preferably comprises a contact surface 640 configured to have a shape and contour that is designed to correspond to and to fit the implant receiving surface 420 on the metatarsal head 410. The metatarsal drill guide 600 is also preferably arranged to use the same nails, rivets, wires or similar attachment means as was used for the metatarsal saw guide 500, so that unnecessary drilling of holes in the metatarsal bone 400 can be avoided.

The metatarsal saw guide 500 and/or the metatarsal drill guide 600 may comprise a rotational position indicator, which may be used to make a marking on the cartilage or bone at the side of the implant receiving surface 420 on the metatarsal head 410. Such a marking may then be used to correctly rotate the metatarsal implant 300 before the metatarsal implant 300 is positioned on the implant receiving surface 420 on the metatarsal head 410. If the metatarsal implant 300 comprises a positioning mark 350, the alignment of this positioning mark 350 with the marking at the side of the implant receiving surface 420 on the metatarsal head 410 ensures that the metatarsal implant 300 is correctly positioned on the implant receiving surface 420. The metatarsal saw guide 500 and/or the metatarsal drill guide 600 is then preferably configured to allow such a marking to be made while the metatarsal saw guide 500 and/or the metatarsal drill guide 600 is attached to the metatarsal bone 400. The metatarsal saw guide 500 and/or the metatarsal drill guide 600 may for this purpose comprise an indentation at the position of the rotational position indicator on the metatarsal saw guide 500 and/or the metatarsal drill guide 600. The marking may e.g. be added to the cartilage surface by inserting a marking pen into the indentation in the metatarsal saw guide 500 and/or the metatarsal drill guide 600 when the metatarsal saw guide 500 and/or the metatarsal drill guide 600 is attached to the metatarsal bone 400. However, the metatarsal implant 300 may also comprise a positioning mark 350 that is simply indication of a direction in relation to the anatomy of the joint. In this case, no marking on the cartilage or bone is needed.

A correct rotational positioning of the metatarsal implant 300 is important because the articulating surface 310 of the metatarsal implant 300 will in most situations not be rotationally symmetric. An important reason for designing the articulating surface 310 of the metatarsal implant 300 to match the simulated healthy articulating surface of the metatarsal head 410 is to ensure that the metatarsal implant 300 fits smoothly on the metatarsal head 410 and takes full account of the sesamoid bones. If the metatarsal implant 300 is not attached in the correct rotational position, there is a risk that the sesamoid bones will lock against the metatarsal implant 300, and that the patient will thus not be given back a full range of motion.

A marking on the cartilage surface makes it easy for the surgeon to attach the metatarsal implant 300 to the metatarsal head 410 with a correct rotational positioning, if the metatarsal implant 300 also comprises a positioning mark 350. However, the metatarsal implant 300 may also comprise a positioning mark 350 that is simply indication of a direction in relation to the anatomy of the joint.

Insert tools may be used to aid the positioning of the metatarsal implant 300 on the implant receiving surface 420 of the metatarsal head 410. It is e.g. possible to use a mandrel as an insert tool, as is commonly known for trochlear implants. There may be a positioning mark on the insert tool, so that the implant engaging portion may be correctly rotated with respect to the metatarsal implant 300.

In order to repair the damaged cartilage in the metatarsal head 410, a metatarsal surgical kit comprising the above described metatarsal implant 300, the above described at least one metatarsal saw guide 500, the above described metatarsal drill guide 600, and potentially also an insert tool may be used. Even if the metatarsal implant 300 is an implant selected from a predefined set of standardized implants having varying dimensions, it is still preferred to use a customized metatarsal saw guide 500, having a contact surface 540 configured to have a shape and contour that is designed to correspond to and to fit the actual contour of the surface of the metatarsal bone 400 in a predetermined area of the metatarsal bone 400, since this will ensure that the metatarsal saw guide 500 will have a stable mounting in the correct position on the metatarsal bone 400. This helps ensuring that the implant receiving surface 420 will be created in the exact position of the determined damage. However, the metatarsal drill guide 600 may be standardized, since its contact surface 640 need only be adapted to the implant receiving surface 420, which for standardized implants is preferably standardized.

When there is damage in both the proximal phalanges 220 and the metatarsal head 410, a metatarsophalangeal surgical kit may instead be used. The metatarsophalangeal surgical kit may comprise the above described metatarsophalangeal implant arrangement 200, the above described at least one metatarsal saw guide 500, a phalangeal guide tool for the phalangeal implant 250, and one or more insert tools. The metatarsal saw guide 500 for the metatarsal implant and/or the phalangeal guide tool for the phalangeal implant 250 preferably comprise visual markings, so that they are visually distinct from each other. In this way, it will be clear to the surgeon which guide tool to use for which implant. The phalangeal guide tool may be similar to the above described metatarsal saw guide 500, or it may instead be a drill guide, depending on the shape of the phalangeal implant 250.

The insert tools may be one or more mandrels to aid positioning of the implants 250, 300. A surgical kit may also comprise further instruments, such as e.g. an implant dummy 650 (as illustrated in FIG. 6a) for verifying that the implant receiving surface 420 has the correct size and shape for receiving the metatarsal implant 300 before attachment of the implant. For a metatarsophalangeal surgical kit, it is advantageous if either all the instruments associated with the phalangeal implant 250 or all the instruments associated with the metatarsal implant 300 comprise visual markings, so that it is visually clear which instruments belong together. The visual markings may be any type of markings that are visible to a surgeon, such as e.g. color markings. However, the visual markings may also be tactile, such as ribs, recesses or indentations on the parts of the instruments that are held by the surgeon during use.

### METHOD EMBODIMENTS

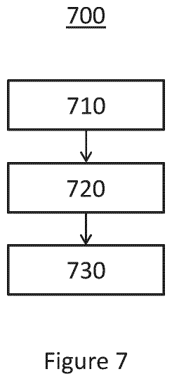

FIG. 7 is a flow diagram of embodiments of a method 700 for customizing a metatarsal implant 300 for repairing damage in a metatarsophalangeal joint of a patient, the metatarsal implant 300 being adapted to be attached to an implant receiving surface 420 on a metatarsal head 410 of the patient. In accordance with one or more embodiments, the method 700 comprises:

Step 710: obtaining a three-dimensional image representation of the metatarsal head 410 based on medical images generated using a medical imaging system 130.

Step 720: determining damage to the metatarsal head 410 by analyzing medical images generated using a medical imaging system 130.

Step 730: determining the shape and dimensions of a customized metatarsal implant 300 suitable for repairing said determined damage, using said three-dimensional image representation of the metatarsal head 410.

This enables the customizing of a metatarsal implant that takes full account of the sesamoid bones, and may also extend far enough to always interact with the sesamoid bones, even when the toe is straight. The metatarsophalangeal joint is preferably the first metatarsophalangeal joint, but other metatarsophalangeal joints of a patient are also conceivable.

In embodiments, the determining 730 of the shape and dimensions of the customized metatarsal implant 300 involves simulating a healthy articulating surface of the metatarsal head 410 at the site of the determined damage, including designing the surface of the customized metatarsal implant 300 to match said simulated healthy articulating metatarsal surface.

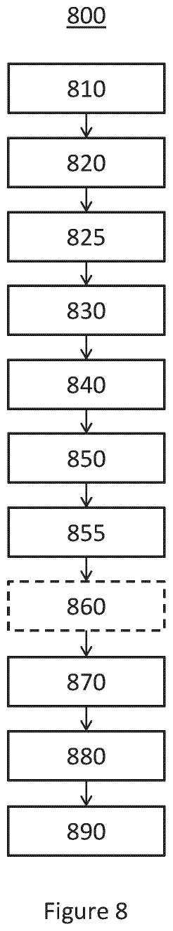

FIG. 8 is a flow diagram of embodiments of a method for attaching a metatarsal implant 300 to an implant receiving surface 420 which has been created on a metatarsal head 410 by sawing off sections of the metatarsal head 410, for repairing damage in a metatarsophalangeal joint of a patient. In accordance with one or more embodiments, the method 800 comprises:

Step 810: attaching at least one metatarsal saw guide 500 to the metatarsal bone 400, the at least one metatarsal saw guide 500 comprising a contact surface 540 configured to have a shape and contour that is designed to correspond to and to fit the actual contour of the metatarsal bone 400 in a predetermined area of the metatarsal bone 400.

Step 820: creating an implant receiving surface 420 on the metatarsal head 410 by sawing the implant receiving surface 420, using the at least one metatarsal saw guide 500.

Step 825: removing the at least one metatarsal saw guide 500 from the metatarsal bone 400.

Step 830: using a metatarsal implant dummy 650 for verifying that the implant receiving surface 420 has the correct size and shape for receiving the metatarsal implant 300.

Step 840: attaching a metatarsal drill guide 600 to the metatarsal bone 400, the metatarsal drill guide 600 comprising a contact surface 640 configured to have a shape and contour that is designed to correspond to and to fit the implant receiving surface 420 on the metatarsal head 410.

Step 850: drilling a recess for an implant peg 320 extending from a bone contacting surface 330 of the metatarsal implant 300.

Step 855: removing the metatarsal drill guide 600 from the metatarsal bone 400.

Step 870: placing the metatarsal implant 300 on the implant receiving surface 420.

Step 880: pressing the metatarsal implant 300 to the implant receiving surface 420 using an insert tool.

Step 890: removing the insert tool.

The metatarsophalangeal joint is preferably the first metatarsophalangeal joint, but other metatarsophalangeal joints of a patient are also conceivable.

In embodiments, the method 800 further comprises:

Step 860: applying an adhesive, such as e.g. bone cement, on the implant receiving surface 420, and/or on a bone contacting surface 330 of the metatarsal implant 300, before placing the metatarsal implant 300 on the implant receiving surface 420.

In embodiments, the method 800 further comprises making a marking on the cartilage at the side of the implant receiving surface 420 on the metatarsal head 410, in order to ensure a correct rotational positioning of the implant.

### Further Embodiments

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually. Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

1. A metatarsal implant for repairing damage in a metatarsophalangeal joint of a patient, the metatarsal implant being adapted to be attached to an implant receiving surface which has been created on a metatarsal head of the patient by sawing off sections of the metatarsal head, wherein a bone contacting surface of the metatarsal implant is designed to correspond to the implant receiving surface, and an articulating surface of the metatarsal implant is designed to correspond to the curvature of a simulated healthy articulating surface of the damaged metatarsal head at a site of diseased cartilage and/or bone, where the contour curvature of the articulating surface is generated based on a determined surface curvature of the cartilage and/or the bone in a predetermined area at the site of diseased cartilage and/or bone, to mimic the original, undamaged, articulating surface of the metatarsal head.

2. The metatarsal implant according to claim 1, further comprising an implant peg extending from a bone contacting surface of the metatarsal implant.

3. The metatarsal implant according to claim 1, wherein the articulating surface of the metatarsal implant comprises a positioning mark.

4. A metatarsophalangeal implant arrangement for repairing damage in a metatarsophalangeal joint of a patient, the metatarsophalangeal implant arrangement comprising a phalangeal implant, comprising an articulating surface, and a metatarsal implant, adapted to be attached to an implant receiving surface which has been created on a metatarsal head by sawing off sections of the metatarsal head, where a bone contacting surface of the metatarsal implant is designed to correspond to the implant receiving surface, and the articulating surfaces of the phalangeal implant and the metatarsal implant are designed to allow that they interact with each other when the implants are implanted into the metatarsophalangeal joint of the patient, wherein:
the articulating surface of the metatarsal implant is a metal, metal alloy or ceramic surface; and
the articulating surface of the phalangeal implant is not a metal, metal alloy or ceramic surface.

5. The metatarsophalangeal implant arrangement according to claim 4, wherein the articulating surface of the metatarsal implant comprises titanium (Ti) or titanium alloy, titanium nitride (TiN) titanium niobium nitride (TiNbN), and/or a cobalt-chromium (CoCr) alloy.

6. The metatarsophalangeal implant arrangement according to claim 4, wherein the articulating surface of the phalangeal implant comprises a polymer material.

7. The metatarsophalangeal implant arrangement according to claim 4, wherein the phalangeal implant comprises a bone contacting surface comprising titanium (Ti) or titanium alloy, titanium nitride (TiN), titanium niobium nitride (TiNbN), and/or a cobalt-chromium (CoCr) alloy.

8. The metatarsophalangeal implant arrangement according to claim 4, wherein the metatarsal implant is adapted to be attached to the implant receiving surface which has been created on the metatarsal head of the patient by sawing off sections of the metatarsal head, wherein the bone contacting surface of the metatarsal implant is designed to correspond to the implant receiving surface, and wherein the articulating surface of the metatarsal implant is designed to correspond to the curvature of a simulated healthy articulating surface of the damaged metatarsal head at a site of diseased cartilage and/or bone, where the contour curvature of the articulating surface is generated based on a determined surface curvature of the cartilage and/or the bone in a predetermined area at the site of diseased cartilage and/or bone, to mimic the original, undamaged, articulating surface of the metatarsal head.

9. A metatarsal surgical kit, comprising:
the metatarsal implant according to claim 1;
at least one metatarsal saw guide comprising a contact surface configured to have a shape and contour that is designed to correspond to and to fit the actual contour of the metatarsal bone in a predetermined area of the metatarsal bone; and
a metatarsal drill guide for drilling a recess for an implant peg extending from a bone contacting surface of the metatarsal implant, the metatarsal drill guide comprising a contact surface configured to have a shape and contour that is designed to correspond to and to fit an implant receiving surface which has been created on a metatarsal head by sawing off sections of the metatarsal head.

10. The metatarsal surgical kit according to claim 9, further comprising an insert tool configured to be used for attaching the metatarsal implant to the implant receiving surface on the metatarsal head, wherein the insert tool has an implant engaging portion that has a surface curvature that substantially corresponds to the surface curvature of the articulating surface of the metatarsal implant.

11. The metatarsal surgical kit according to claim 9 wherein the at least one metatarsal saw guide comprises one or more contact surface extensions that extend around at least a part of the metatarsal head.

12. A metatarsophalangeal surgical kit comprising:
the metatarsophalangeal implant arrangement according to claim 4;
at least one metatarsal saw guide comprising a contact surface configured to have a shape and contour that is designed to correspond to and to fit the actual contour of the metatarsal bone in a predetermined area of the metatarsal bone;
a phalangeal guide tool comprising a contact surface configured to have a shape and contour that is designed to correspond to and to fit the actual contour of the bone in a predetermined area of the proximal phalanges; and
one or more insert tools, configured to be used for attaching the metatarsal implant to an implant receiving surface on a metatarsal head, and/or
attaching the phalangeal implant to an implant receiving surface on a proximal phalanges,
wherein the saw guide for the metatarsal implant and/or the guide tool for the phalangeal implant comprise visual markings, so that they are visually distinct from each other.

13. The metatarsophalangeal surgical kit according to claim 12, wherein the at least one metatarsal saw guide comprises one or more contact surface extensions that extend around at least a part of the metatarsal head.

14. A metatarsal saw guide comprising a contact surface configured to have a shape and contour that is designed to correspond to and to fit the actual contour of the metatarsal bone in a predetermined area of the metatarsal bone, wherein the metatarsal saw guide comprises a plurality of different saw blade guides.

15. The metatarsal saw guide according to claim 14, further comprising one or more contact surface extensions that extend around at least a part of the metatarsal head.

16. A system for customizing a metatarsal implant for repairing damage in a metatarsophalangeal joint of a patient, the metatarsal implant being adapted to be attached to an implant receiving surface which has been created on a metatarsal head of the patient by sawing off sections of the metatarsal head, the system comprising at least one processor configured to:
obtain a three-dimensional image representation of the metatarsophalangeal joint of the patient based on medical images generated using a medical imaging system;
determine damage to the metatarsophalangeal joint of the patient by analyzing medical images generated using a medical imaging system; and
determine the shape and dimensions of a customized metatarsal implant suitable for repairing said determined damage, using said three-dimensional image representation of the metatarsophalangeal joint, by designing a bone contacting surface of the metatarsal implant to correspond to the implant receiving surface, and generating the contour curvature of the articulating surface based on the determined surface curvature of the cartilage and/or the bone in a predetermined area at the site of diseased cartilage and/or bone, to mimic the original, undamaged, articulating surface of the metatarsal head.

17. The system according to claim 16, wherein the at least one processor is configured to determine the shape and dimensions of the customized metatarsal implant by simulating a healthy articulating metatarsal surface at the site of the determined damage, including designing the surface of the customized metatarsal implant to match said simulated healthy articulating metatarsal surface.

18. A method for customizing a metatarsal implant for repairing damage in a metatarsophalangeal joint of a patient, the metatarsal implant being adapted to be attached to an implant receiving surface which has been created on a metatarsal head of the patient by sawing off sections of the metatarsal head, the method comprising:
obtaining a three-dimensional image representation of the metatarsophalangeal joint based on medical images generated using a medical imaging system;
determining damage to the metatarsophalangeal joint by analyzing medical images generated using a medical imaging system; and
determining the shape and dimensions of a customized metatarsal implant suitable for repairing said determined damage, using said three-dimensional image representation of the metatarsophalangeal joint, by designing a bone contacting surface of the metatarsal implant to correspond to the implant receiving surface, and generating the contour curvature of the articulating surface based on the determined surface curvature of the cartilage and/or the bone in a predetermined area at the site of diseased cartilage and/or bone, to mimic the original, undamaged, articulating surface of the metatarsal head.

19. The method according to claim 18, wherein the determining of the shape and dimensions of the customized metatarsal implant involves simulating a healthy articulating metatarsal surface at the site of the determined damage, including designing the surface of the customized metatarsal implant to match said simulated healthy articulating metatarsal surface.

20. A non-transitory machine-readable medium on which is stored machine-readable code which, when executed by at least one processor, controls the processor to perform the method of claim 18.

21. A method for attaching a metatarsal implant to an implant receiving surface on a metatarsal head, for repairing damage in a metatarsophalangeal joint of a patient, the method comprising:
attaching at least one metatarsal saw guide to the metatarsal bone, the metatarsal saw guide comprising a contact surface configured to have a shape and contour that is designed to correspond to and to fit the actual contour of the metatarsal bone in a predetermined area of the metatarsal bone;
creating an implant receiving surface on the metatarsal head by sawing off sections of the metatarsal head, using the at least one metatarsal saw guide;
removing the at least one metatarsal saw guide from the metatarsal bone; using a metatarsal implant dummy for verifying that the implant receiving surface has the correct size and shape for receiving the metatarsal implant;
attaching a metatarsal drill guide to the metatarsal bone, the metatarsal drill guide comprising a contact surface configured to have a shape and contour that is designed to correspond to and to fit the implant receiving surface on the metatarsal head;
drilling a recess for an implant peg extending from a bone contacting surface of the metatarsal implant;
removing the metatarsal drill guide from the metatarsal bone;
placing the metatarsal implant on the implant receiving surface on the metatarsal head;
pressing the metatarsal implant to the implant receiving surface using an insert tool; and
removing the insert tool.

22. The method according to claim 21, further comprising applying an adhesive on the implant receiving surface, and/or on a bone contacting surface of the metatarsal implant, before placing the metatarsal implant on the implant receiving surface.
